(19) United States
(12) Patent Application Publication
(10) Pub. No.: US 2018/0057420 A1
(43) Pub. Date: Mar. 1, 2018

(54) PROCESSES AND COMPOSITIONS FOR TOLUENE METHYLATION IN AN AROMATICS COMPLEX

(71) Applicant: UOP LLC, Des Plaines, IL (US)

(72) Inventors: Antoine Negiz, Wilmette, IL (US); Elie Jean Fayad, Al Khobar (SA); Ali Jahel, Al Khobar (SA); Sulaiman Saleh Al-Khattaf, Dhahran (SA); Abdullah Mohammed Aitani, Dhahran (SA); Palani Arudra, Dhahran (SA); Atif Fazal, Dhahran (SA)

(21) Appl. No.: 15/796,724

(22) Filed: Oct. 27, 2017

Related U.S. Application Data
(63) Continuation of application No. PCT/US2016/061025, filed on Nov. 9, 2016.
(60) Provisional application No. 62/259,954, filed on Nov. 25, 2015.

(51) Int. Cl. C07C 2/86; C07C 5/22; C07C 6/12; C07C 7/12; C07C 15/08; B01J 29/40

(52) U.S. Cl. CPC C07C 2/864; C07C 5/222; C07C 6/126; C07C 7/12; B01J 2208/00539; B01J 29/40; C07C 2529/40; B01J 2208/00017; C07C 15/08

(57) ABSTRACT

This present disclosure relates to processes and compositions for toluene methylation in an aromatics complex for producing paraxylene. More specifically, the present disclosure relates to a process for producing paraxylene which includes alkylating a toluene stream and a methanol stream in a toluene methylation zone operating under toluene methylation conditions in the presence of a catalyst comprising a MFI crystal to produce a toluene methylation product stream.

### CROSS-REFERENCE TO RELATED APPLICATIONS

This application is a Continuation of copending International Application No. PCT/US2016/061025 filed Nov. 9, 2016 which application claims benefit of U.S. Provisional Application No. 62/259,954 filed Nov. 25, 2015, now expired, the contents of which cited applications are hereby incorporated by reference in their entirety.

### FIELD

This present disclosure relates to processes and compositions for toluene methylation in an aromatics complex for producing paraxylene. More specifically, the present disclosure relates to a process for producing paraxylene which includes alkylating a toluene stream and a methanol stream in a toluene methylation zone operating under toluene methylation conditions in the presence of a catalyst comprising crystals with the MFI framework topology referred to hereafter as MFI crystal(s) to produce a toluene methylation product stream.

### BACKGROUND

The xylene isomers are produced in large volumes from petroleum as feedstocks for a variety of important industrial chemicals. The most important of the xylene isomers is para-xylene, the principal feedstock for polyester, which continues to enjoy a high growth rate from large base demand. Ortho-xylene is used to produce phthalic anhydride, which supplies high-volume but relatively mature markets. Meta-xylene is used in lesser but growing volumes for such products as plasticizers, azo dyes and wood preservers. Ethylbenzene generally is present in xylene mixtures and is occasionally recovered for styrene production, but is usually considered a less-desirable component of C₈ aromatics.

Among the aromatic hydrocarbons, the overall importance of xylenes rivals that of benzene as a feedstock for industrial chemicals. Xylenes and benzene are produced from petroleum by reforming naphtha but not in sufficient volume to meet demand, thus conversion of other hydrocarbons is necessary to increase the yield of xylenes and benzene. Often toluene is de-alkylated to produce benzene or selectively disproportionated to yield benzene and C₈ aromatics from which the individual xylene isomers are recovered.

An aromatics complex flow scheme has been disclosed by Meyers in the HANDBOOK OF PETROLEUM REFINING PROCESSES, 2d. Edition in 1997 by McGraw-Hill, and is incorporated herein by reference.

Traditional aromatics complexes send toluene to a transalkylation zone to generate desirable xylene isomers via transalkylation of the toluene with A₉₊ components. A₉₊ components are present in both the reformate bottoms and the transalkylation effluent.

Prior art processes that are used to convert aromatic compounds utilize conditions that require high concentrations of hydrogen in the feedstock and also require the recycling of hydrogen and other gases during the conversion process, which renders these processes expensive and cost-inefficient. Thus, there is a need for an energy-efficient process that converts aromatic compounds to xylene compounds via methylation that does not require the recycling of hydrogen or other gases.

### SUMMARY

The present subject matter relates to processes and compositions for toluene methylation in an aromatics complex for producing paraxylene. A first embodiment of the invention is a process for producing paraxylene, comprising alkylating a toluene stream and a methanol stream in a toluene methylation zone operating under toluene methylation conditions in the presence of a catalyst comprising an MFI crystal to produce a toluene methylation product stream. An embodiment of the invention is one, any or all of prior embodiments in this paragraph up through the first embodiment in this paragraph, wherein the catalyst includes MFI crystals with a framework silica to alumina ratio of about 50 to about 10,000, more preferably about 100 to about 6,000, or even more preferably about 500 to about 3,000. An embodiment of the invention is one, any or all of prior embodiments in this paragraph up through the first embodiment in this paragraph, wherein the toluene methylation conditions include a temperature of about 250° C. to about 750° C., more preferably between 350° C. and 650° C., even more preferably between 400° C. and 600° C. An embodiment of the invention is one, any or all of prior embodiments in this paragraph up through the first embodiment in this paragraph, wherein the toluene methylation conditions include a pressure of about 3 Barg to about 250 Barg. An embodiment of the invention is one, any or all of prior embodiments in this paragraph up through the first embodiment in this paragraph, wherein the toluene methylation product stream has a benzene to total xylene molar ratio of less than 1, or preferably less than 0.5, or more preferably less than 0.1. An embodiment of the invention is one, any or all of prior embodiments in this paragraph up through the first embodiment in this paragraph, wherein the catalyst includes MFI crystals with a framework silica to alumina ratio of 2000.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### Definitions

As used herein, the term “stream”, “feed”, “product”, “part” or “portion” can include various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. Each of the above may also include aromatic and non-aromatic hydrocarbons.

Hydrocarbon molecules may be abbreviated C₁, C₂, C₃, Cₙ where “n” represents the number of carbon atoms in the one or more hydrocarbon molecules or the abbreviation may be used as an adjective for, e.g., non-aromatics or compounds. Similarly, aromatic compounds may be abbreviated A₆, A₇, A₈, and where “n” represents the number of carbon atoms in the one or more aromatic molecules. Furthermore, a superscript “+” or “−” may be used with an abbreviated one or more hydrocarbons notation, e.g., C₃⁺ or C₃⁻, which is inclusive of the abbreviated one or more hydrocarbons. As an example, the abbreviation “C₃⁺” means one or more hydrocarbon molecules of three or more carbon atoms.

As used herein, the term “zone” can refer to an area including one or more equipment items and/or one or more sub-zones. Equipment items can include, but are not limited to, one or more reactors or reactor vessels, separation vessels, distillation towers, heaters, exchangers, pipes, pumps, compressors, and controllers. Additionally, an equipment item, such as a reactor, dryer, or vessel, can further include one or more zones or sub-zones.

As used herein, the term “rich” can mean an amount of at least generally 50%, and preferably 70%, by mole, of a compound or class of compounds in a stream.

As used herein, the term “silica to alumina ratio” can mean the molar ratio of silicon and aluminum species.

As used herein, the term “MFI crystal” can mean a crystallized microporous solid displaying an X-ray diffraction pattern characteristic of the MFI framework type as defined by the International Zeolite Association—Structure Commission (http://www.iza-structure.org/databases/)

### BRIEF DESCRIPTION OF THE DRAWINGS

FIG. 1 graphically illustrates X-Ray diffraction patterns for samples a) Catalyst B; b) Catalyst A; d) Catalyst C; and e) Catalyst D.

FIG. 2 graphically illustrates OH-IR Region Spectra for Catalysts A, B, C, and D.

FIG. 3 graphically illustrates NH3-IR Region Spectra for Catalysts A, B, C, and D.

Corresponding reference characters indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present disclosure. Also, common but well-understood elements that are useful or necessary in a commercially feasible embodiment are often not depicted in order to facilitate a less obstructed view of these various embodiments of the present disclosure.

### DETAILED DESCRIPTION

The following description is not to be taken in a limiting sense, but is made merely for the purpose of describing the general principles of exemplary aspects. The scope of the present disclosure should be determined with reference to the claims.

An embodiment of the invention is directed to a method for producing xylenes comprising the steps of: loading a zeolite catalyst into to a fixed bed reactor system; feeding a feedstock to the fixed bed reactors, wherein the feedstock comprises at least one aromatic compound, methanol and water; reacting the feedstock in the presence of the zeolite catalyst to form an effluent, wherein the effluent comprises water, aromatic hydrocarbons, and light hydrocarbons; cooling the effluent; separating a vapor phase stream from an aqueous stream and a hydrocarbon stream in a separator; distilling the hydrocarbon stream to form a product fraction and a fraction containing unreacted aromatic compounds; recycling a portion of the fraction containing unreacted aromatic compounds and methanol in the aqueous stream to the fixed bed reactors; and diverting the vapor phase stream away from the fixed bed reactor system. In this embodiment, the vapor phase stream or off gas is not recycled back into the feedstock or reactor system.

A mixture of methanol and aromatic compounds are fed into methylation reactors containing a zeolite catalyst. The effluent that is formed in the methylation reactors is fed into a separator where a vapor phase stream, an aqueous phase stream and a hydrocarbon phase stream are separated. The hydrocarbon phase stream is fed into a distillation section to form a product fraction comprising xylenes. The unreacted aromatic fraction is fed back into reactor system. In certain embodiments of the invention, an unreacted methanol fraction is removed from the distillation section and is concentrated and fed back into reactor system, along with the water (aqueous stream) in the reactor effluent.

In certain embodiments of the invention, the fixed bed reactor system comprises a single or a plurality of fixed reactors, where the reactors may be arranged in series or parallel.

The reactor system used in the inventive process can be designed in any number of ways to accommodate specific process conditions. In certain embodiments, the reactor system comprises a single shell with a single bed. In other embodiments, the reactor system comprises a single shell having a plurality of beds in which the aromatic compounds and the methanol are fed into the reactor system through different input points. Multiple shell reactor systems connected in series may include the use of a standby shell.

In an embodiment of the invention, the method is carried out at a temperature of about 250° C. to about 750° C., more preferably between about 350° C. and about 650° C., even more preferably between about 400° C. and about 600° C. In other embodiments of the invention, method is carried out at a pressure of about 1 Barg to about 250 Barg, more preferably between about 1 Barg to about 100 Barg, even more preferably between 2 Barg to about 50 Barg. In one embodiment, the toluene methylation product stream has a benzene to total xylene molar ratio of less than 1, or preferably less than 0.5, or more preferably less than 0.1

In an embodiment of the invention, the aromatic compound that is used in the feedstock is selected from the group consisting of benzene, toluene or a mixture benzene and toluene. In certain embodiments of the invention, the feedstock also comprises hydrogen at a H2 to hydrocarbon(s) molar ratio of less than 20c. In certain embodiments of the invention, the aromatic compound(s) in the feedstock are presentation at a concentration of 30 wt % to 99.5 wt %.

In embodiments of the invention, the zeolite catalyst that is used is a 10-membered ring crystalline microporous solid, hereafter termed zeolite with an MFI framework topology and a defined framework silica to alumina ratio. Note that in this application, the terms zeolite and/or catalyst can be used interchangeably.

In one embodiment, the catalyst includes MFI crystals with a framework silica to alumina ratio of 2000. The catalyst may be comprised of MFI crystals whose sizes include a first largest dimension, a second largest dimension, and a third largest dimension wherein the first largest dimension is at least 20 microns. In one embodiment, the MFI crystal size includes a second dimension of at least 30% of the first dimension, a third dimension of at least 30% of the second dimension. Further, the composition includes at least 60% of the acid sites are that are Bronsted acid sites, wherein at least 70% of the Bronsted acidity is present as weak acid sites, and among the Bronsted acid sites, less than 5% of those exist as strong acid sites.

The claimed process may achieve a paraxylene to xylene selectivity of at least 80/%, about 15% toluene conversion, and a net total xylene yield of close to 15%.

In some embodiments of the invention, the zeolite catalyst is regenerated upon completion of the end of the run of the xylene production process. In some embodiments, the zeolite catalyst is regenerated in situ within the fixed bed reactor system by oxidation. In certain embodiments of the invention, the oxidation process is carried out using a stream of diluted oxygen.

In an embodiment of the invention, the feedstock comprises at least one aromatic compound and methanol in a aromatic compound to methanol molar ratio ranging from 1 to 100. In some embodiments, the ratio range from 1 to 20 and 1 to 5.

In an embodiment of the invention, the product fraction comprises a mixture of xylenes that are present at 30 wt % to 99 wt % of the product fraction, and more preferably at 80 wt % to 95 wt % of the product fraction. The paraxylene selectivity in the mixed xylenes is higher than 70 wt % and more preferably higher than 90 wt %.

In an embodiment of the invention, the conversion of the aromatic compounds in the feedstock obtained using the claimed method ranges from 1 wt % to 50 wt % and more preferably from 5 wt % to 33 wt %. In certain embodiments of the invention, the conversion of the aromatic compounds in the feedstock ranges from 5 wt % to 15 wt %.

### EXAMPLES

The following examples are intended to further illustrate the subject embodiments. These illustrations of different embodiments are not meant to limit the claims to the particular details of these examples.

### Example 1

Catalysts A and B as described in this invention have the MFI crystalline framework and they are crystallized according to the following general description:

A mixture in water or any other suitable crystallization medium of at least one source of Aluminum, at least one source of Silicon, at least one source of a suitable structure directing agent (SDA), at least one source of fluoride acting as mineralizer. The resulting mixture is heated at a set temperature until the formation of the MFI type crystals.

According to this invention, the MFI framework is crystallized from a mixture containing at least one source of Aluminum. Aluminum sources include, but not limited to sodium aluminate (NaAlO₂), aluminium sulfate (Al₂(SO₄)₃), aluminium nitrate (Al(NO₃)₃. 9H₂O), aluminium hydroxide (Al(OH)₃) and aluminium nitrate nonahydrate (Al(NO₃)₃.9H₂O.

According to this invention, the MFI framework is crystallized from a mixture containing at least one source of Silicon. Silicon sources include, but not limited to fumed silica, colloidal silica, precipitated silica and sodium meta silicate.

According to this invention, the MFI framework is crystallized from a mixture containing at least one structure directing agent. Structure directing agents sources include, but not limited to tetrapropylammonium bromide (TPABr), tetrapropylammonium hydroxide (TPAOH), ethylamine, diethylamine, triethylamine and tetraethylamine.

According to this invention, the synthesis mixture comprises one or more mineralizers for crystallizing the MFI crystals, amongst said mineralizer(s) at least one comprises fluoride in its molecular formula. Fluoride as mineralizer sources include, but not limited to NH₄F, NH₄HF₂, HF, (NH₄)₂SiF₆ and AF₃.H₂O. According to this invention and as described in the subsequent claims, using fluoride as a mineralizer to crystallize the MFI crystals is shown to have a positive impact on the shape selectivity of the resulting catalyst. (S. A. Axon and J. Klinowski, APPLIED CATALYSIS A: General, 81 (1992), 27-34). (J. Cejka and B. Wichtorlova, CATALYSIS REVIEWS, 44 (3), 375-421 (2002)).

Catalyst A:

A typical procedure for the Catalyst A synthesis comprises the following: 0.075 g of aluminium nitrate nonahydrate (Al(NO₃)₃.9H₂O, BDH), 4.26 g tetrapropylammonium bromide (TPABr, Fluka) and 11.8518 g ammonium fluoride (NH₄F, Sigma Aldrich) are dissolved into 72 ml of water. Then, 12 g of fumed silica (S5505, particle size 0.014 μm, surface area 200±25 m2/g, Sigma Aldrich) is added and stirred until a homogeneous gel is formed. The gel is subjected to hydrothermal crystallization process at 200° C. for 2 days. The molar composition of the gel is 1 SiO₂: 0.0005 Al₂O₃: 0.08 TPABr: 1.6 NH₄F: 20 H₂O. The gel is washed with water and dried at 100° C. overnight. The template is removed by calcination at 750° C. for 6 hours in air.

Catalyst B:

Catalyst B was prepared with the molar composition of the gel as 1 SiO₂: 0.0005 Al₂O₃: 0.08 TPABr: 0.1 NH₄F: 20 H₂O. The rest of the preparation method was kept the same as Catalyst A.

Catalyst C:

A typical procedure for the Catalyst C synthesis comprises the following: 0.075 g of aluminium nitrate nonahydrate (Al(NO₃)₃.9H₂O, BDH) dissolved in 8.2 ml of water was mixed with 12 g of fumed silica (S5505, particle size 0.014 μm, surface area 200±25 m²/g, Sigma Aldrich) and 8.64 g of tetrapropylammonium hydroxide (40% TPAOH, Sigma Aldrich). The resulting mixture was crystallized for 4 days at 90° C. The composition of the gel was 1.0 SiO₂: 0.085 TPAOH: 0.0005 Al₂O₃: 3.72 H₂O. After crystallization, the product was mixed with water and centrifuged. The obtained solid was dried at 100° C. for overnight and calcined at 750° C. for 6 hours.

Catalyst D:

Catalyst D represents a comparative catalyst. Catalyst D is a commercially available zeolite from Tosoh. Si/Al₂ ratio as measured was very close to 2000, nominally disclosed as 1500 ratio. Catalyst D was purchased commercially from TOSOH, Japan (890HOA) and calcined at 550° C. for 2 hours.

Catalyst E:

Catalyst E is a comparative catalyst not according to the present invention. Zeolyst CBV 28014, nominally 280 Si/Al₂ ratio small crystal acidic parent MFI. Catalyst E was purchased commercially from Zeolyst International USA (CBV 28014) and calcined at 550° C. for 2 hours.

Catalyst F:

Catalyst F is also a comparative and not according to the present invention. Catalyst F was prepared according to the following recipe. The parent ZSM-5 zeolite with SiO₂/Al₂O₃ ratio of 280 and surface area 425 m²/g was purchased from Zeolyst, International (CBV 28014). The parent material is Catalyst E. The material is calcined at 550° C. for 2 hours with heating rate of 5° C./minute. 30 grams of parent zeolite is suspended in 300 ml of n-hexane (Sigma Aldrich) and the mixture was heated until reflux at 70° C. After 30 minutes stirring, tetraethyl orthosilicate (TEOS, Sigma Aldrich) solution corresponding to a loading of 4 wt % SiO₂ is added and silylation is continued for 2 hours at 70° C. with reflux and stirring. Excess n-hexane is removed by evacuation. Finally, the sample is dried at 100° C. for 24 hours and calcined at 550° C. for 4 hours, with a heating rate of 5° C./minute. After each TEOS deposition, 4 g of catalysts were taken from a batch and tested. Silylation treatment was carried out six times using the same procedure.

One skilled in the art will recognize that starting from the MFI crystals as prepared according to the claimed invention, it is obviously anticipated that modifying the materials of the claimed invention further with modification methods that are publicly known can further increase the target para Xylene selectivity. These modifications are known in the public domain literature, and without being limited to the methods we disclose as a few examples, one skilled in this field of research will immediately recognize and anticipate the application of these methods to as the claimed synthesized MFI according to the claimed invention for the purpose of further increasing the para shape selectivity, and specifically further increase the para-Xylene content in the total Xylenes in the alkylation product.

The shape selectivity of the catalyst of this invention can be modified using at least one modifier in its elemental and/or oxidic form, preferably selected from the elements of Groups IIA, IIIA, IVA, VA, VIA, IIIB, IVB, VB, VIB, VIIB and VIIIB of the Periodic Table (IUPAC version). The catalyst can also be modified to increase its shape selectivity through the application of post-synthesis modifications such as steaming or coking.

Many examples on modifiers used, either alone or in combination with at least one other modifier, to enhance the shape selectivity of the MFI crystals can be found in the literature, such as but not limited to, Phosphorous (W. W. Kaeding, S. A. Butter, J. CATAL. 61 (1980) 155), Boron oxide (ZEOLITES, Volume 12, Issue 4, 1992, Pages 347-350), Lanthanum oxide (Zhang et al. CATAL LETT (2009) 130:355-361, DOI 10.1007/s10562-009-9965-3 and Jun Hui Li et al. ADVANCED MATERIALS RESEARCH (2012) Vol. 629 pp. 381-385), Magnesium (Wei Tan et al. MICROPOROUS AND MESOPOROUS MATERIALS Volume 196, 2014, Pages 18-30), and Silicon (Shourong Zheng et al. JOURNAL OF CATALYSIS 241 (2006) 304-311).

It can be also effective to combine the addition of at least one of these modifiers with a steaming step (Jun Hui Li et al. (ADVANCED MATERIALS RESEARCH (2012) Vol. 629 pp. 381-385), performed before and/or after the addition of the modifier(s), on the crystalline porous crystalline material either alone or in combination with a binder or matrix material.

The catalyst of the invention may also be optionally precoked. The precoking step is preferably carried out by initially utilizing the uncoked catalyst in the toluene methylation reaction, during which coke is deposited on the catalyst surface and thereafter controlled within a desired range, typically from about 1 to about 20 wt % and preferably from about 1 to about 5 wt %, by periodic regeneration by exposure to an oxygen-containing atmosphere at an elevated temperature.

Preparation method of P-modified MFI including treatment with phosphorus-containing compounds can readily be accomplished by contacting the porous crystalline material, either alone or in combination with a binder or matrix material, with a solution of an appropriate phosphorus compound, followed by drying and calcining to convert the phosphorus to its oxide form. Contact with the phosphorus-containing compound is generally conducted at a temperature of about 25° C. and about 125° C. for a time between about 15 minutes and about 20 hours. The concentration of the phosphorus in the contact mixture may be between about 0.01 and about 30 wt %.

After contacting with the phosphorus-containing compound, the porous crystalline material may be dried and calcined to convert the phosphorus to an oxide form. Calcination can be carried out in an inert atmosphere or in the presence of oxygen, for example, in air at a temperature of about 150 to 750° C., preferably about 300 to 500° C., for at least 1 hour, preferably 3-5 hours. Preparation method of Si-modified MFI included treatment with Si containing compounds can readily be accomplished by contacting the porous crystalline material, either alone or in combination with a binder or matrix material, with a solution of an appropriate silicon compound, followed by drying and calcining to convert the silicon to its oxide form.

Contact with the silicon-containing compound can be done at a temperature of about 25° C. and about 125° C. for a time between about 15 minutes and about 20 hours. The contacting procedure can be done my refluxing the mixture containing the crystalline material and the Si compound. The concentration of Si in the contact mixture may be between about 0.01 and about 30 wt % relative to the porous crystalline MFI material.

The porous crystalline material may be dried at a temperature between 10 and 150° C. during 0.5 to 48 hours, and calcined to convert the Si to an oxide form. Calcination can be carried out in an inert atmosphere or in the presence of oxygen, for example, in air at a temperature of about 150 to 750° C., preferably about 300 to 500° C., for at least 1 hour, preferably 3-5 hours. It is also obvious for the person skilled in the art that this procedure can be repeated more than one time on the same porous crystalline MFI material to achieve better results.

The steaming procedure included steaming of the porous crystalline material is effected at a temperature between 400° C. and 1100° C. preferably about 500° C. to about 900, and most preferably about 650° C. to 800° C. for about 10 minutes to about 24 hours, preferably from 30 minutes to 5 hours. Representative phosphorus-containing compounds which may be used to incorporate a phosphorus oxide modifier into the catalyst of the invention include derivatives of groups represented by PX₃, RPX₂, R₂ PX, R₃ P, X₃ PO, (XO₃)PO, (XO)₃ P, R₃ P═O, R₃ P═S, RPO₂, PPS₂, RP(OX)₂, RP(S)(SX)₂, R₂ P(O)OX, R₂ P(S)SX, RP(OX)₂, RP(SX)₂, ROP(OX)₂, RSP(SX)₂, (RS)₂ PSP(SR)₂, and (RO)₂ POP(OR)₂, where R is an alkyl or aryl, such as phenyl radical, and X is hydrogen, R, or halide. These compounds include primary, RPH₂, secondary, R₂PH, and tertiary, R3P, phosphines such as butyl phosphine, the tertiary phosphine oxides, R3PO, such as tributyl phosphine oxide, the tertiary phosphine sulfides, R3PS, the primary, RP(O)(OX)₂, and secondary, R₂P(O)OX, phosphonic acids such as benzene phosphonic acid, the corresponding sulfur derivatives such as RP(S)(SX)₂ and R₂P(S)SX, the esters of the phosphonic acids such as dialkyl phosphonate, (RO)₂P(O)H, dialkyl alkyl phosphonates, (RO)₂P(O)R, and alkyl dialkylphosphinates, (RO)P(O)R₂; phosphinous acids, R₂POX, such as diethylphosphinous acid, primary, (RO)P(OX)₂, secondary, (RO)₂POX, and tertiary, (RO)3P, phosphites, and esters thereof such as the monopropyl ester, alkyl dialkylphosphinites, (RO)PR₂, and dialkyl alkyphosphinite, (RO)₂PR, esters. Corresponding sulfur derivatives may also be employed including (RS)₂P(S)H, (RS)₂P(S)R, (RS)P(S)R₂, R₂PSX, (RS)P(SX)₂. (RS)₂PSX, (RS)₃P, (RS)PR₂, and (RS)PR.

Examples of phosphite esters include trimethylphosphite, triethylphosphite, diisopropylphosphite, butylphosphite, and pyrophosphites such as tetraethylpyrophosphite. The alkyl groups in the mentioned compounds preferably contain one to four carbon atoms. Other suitable phosphorus-containing compounds include ammonium hydrogen phosphate, the phosphorus halides such as phosphorus trichloride, bromide, and iodide, alkyl phosphorodichloridites, (RO)PCI₂, dialkylphosphoro-chloridites, (RO)₂PCI, dialkylphosphinochloroidites, R₂PCI, alkyl alkylphosphonochloridates, (ROXR)P(O)CI, dialkyl phosphinochloridates, R₂P(O)CI, and RP(O)CI. Applicable corresponding sulfur derivatives include (RS)PCI₂, (RS)₂PCI, (RS)(R)P(S)CI, and R₂P(S)CI. Particular phosphorus-containing compounds include ammonium phosphate, ammonium dihydrogen phosphate, diammonium hydrogen phosphate, diphenyl phosphine chloride, trimethylphosphite, phosphorus trichloride, phosphoric acid, phenyl phosphine oxychloride, trimethylphosphate, diphenyl phosphinous acid, diphenyl phosphinic acid, diethylchlorothiophosphate, methyl acid phosphate, and other alcohol-P₂O5 reaction products

Similar techniques known in the art can be used to incorporate other modifying oxides into the catalyst of the invention, such as but not limited to Boron oxide, Lanthana, Magnesia. Representative boron-containing compounds which may be used to incorporate a boron oxide modifier into the catalyst of the invention include boric acid, trimethylborate, boron oxide, boron sulfide, boron hydride, butylboron dimethoxide, butylboric acid, dimethylboric anhydride, hexamethylborazine, phenyl boric acid, triethylborane, diborane and triphenyl boron. Representative magnesium-containing compounds include magnesium acetate, magnesium nitrate, magnesium benzoate, magnesium propionate, magnesium 2-ethylhexoate, magnesium carbonate, magnesium formate, magnesium oxylate, magnesium bromide, magnesium hydride, magnesium lactate, magnesium laurate, magnesium oleate, magnesium palmitate, magnesium salicylate, magnesium stearate and magnesium sulfide. Representative lanthanum-containing compounds include lanthanum acetate, lanthanum acetylacetonate, lanthanum carbonate, lanthanum chloride, lanthanum hydroxide, lanthanum nitrate, lanthanum phosphate and lanthanum sulfate.

### Example 2

Catalysts A, B, C, D were tested for MeOH and toluene alkylation reaction. Feed 2:1 (mol:mol) Tol:MeOH, H2:HC(HC=Tol+MeOH)=4 (mol:mol); WHSV (on 1 gram of active catalyst)=2, and reactor pressure of 3 Barg. Testing details are as follows.

The toluene methylation reaction was performed in a fixed-bed tubular reactor (stainless steel tube grade 316 material, 7.92 mm ID×14.27 mm OD×203.2 mm length) packed with 1 g of catalyst mixed with 5 g of silicon carbide as diluent. Before catalytic run, the catalyst was activated in a hydrogen stream at 550° C. for 1 hour and 0 Barg reactor pressure, and then a mixture of the hydrocarbon feed (toluene and methanol) and hydrogen was passed through the catalyst bed. The reaction was carried out at 450° C., 500° C. and 550° C. (3 Barg, 2 WHSV, H2:HC=4:1). The reactor pressure was controlled with a reactor effluent BPR at 3 Barg (43.51 psig) maximum. The liquid product was collected at room temperature and analyzed with gas chromatography equipped with Innovax column, which is capable of separating pX from the rest of the Xylenes and measure aromatics composition. MeOH cannot be found in the liquid samples at the test conditions. This test is used to simply screen materials having high pX/X selectivity. Table 1 illustrates the test results.

TABLE 1

| | Time on | Toluene | Xylene in | Paraxylene | Reactor |
|---|---|---|---|---|---|
| | Stream, | conversion, | total product, | in total | Temperature |
| Catalyst | hrs. | % | wt % | Xylene, % | ABT, ° C. |
| A | 1 | 25 | 19 | 92 | 450 |
| A | 2 | 26 | 19 | 91 | 450 |
| B | 1 | 20 | 15 | 82 | 450 |
| B | 2 | 23 | 17 | 76 | 450 |
| C | 1 | 41 | 36 | 25 | 450 |
| C | 2 | 42 | 36 | 24 | 450 |
| D | 1 | 30 | 25 | 26 | 450 |
| D | 2 | 33 | 26 | 25 | 450 |
| A | 3 | 24 | 19 | 88 | 500 |
| A | 4 | 23 | 20 | 90 | 500 |
| B | 3 | 22 | 19 | 71 | 500 |
| B | 4 | 23 | 19 | 70 | 500 |
| C | 3 | 42 | 37 | 26 | 500 |
| C | 4 | 34 | 31 | 27 | 500 |
| D | 3 | 37 | 31 | 24 | 500 |
| D | 4 | 36 | 31 | 24 | 500 |
| A | 5 | 27 | 24 | 85 | 550 |
| A | 6 | 26 | 24 | 86 | 550 |
| B | 5 | 16 | 14 | 73 | 550 |
| B | 6 | 14 | 11 | 70 | 550 |
| C | 5 | 29 | 27 | 29 | 550 |
| C | 6 | 26 | 25 | 30 | 550 |
| D | 5 | 30 | 26 | 24 | 550 |
| D | 6 | 26 | 23 | 24 | 550 |

As Table 1 clearly illustrates, Catalysts A and B have high percent paraxylene in total/xylene, greater than 70% selectivity, more preferably greater than 80% selectivity, yet more preferably greater than 90% selectivity. In comparison, Catalysts C and D represent close to equilibrium percent paraxylene in total/xylene at less than 30%.

### Example 3

Catalysts A, B, C, and D as prepared were subjected to scanning electron microscopy (SEM). SEM images were taken with a JEOL JSM-5800 scanning microscope. Before taking SEM photographs, the samples were loaded onto a sample holder, held with conductive aluminum tape, and coated with a film of gold in vacuum using a cressington sputter ion-coater for 20 s with 15 mA current.

A SEM Image of Catalyst A was created. One or more crystal particles with the following three-dimensional size in microns, at least 30 microns for the longest dimension, at least 8 microns for the second longest dimension, and at least 4 microns for the third longest dimension. More specifically, the Catalyst A SEM shows at least one crystal particle observed to have the following three-dimensional crystal particle size with 33.89 microns as its longest dimension, 8.15 microns in its second longest dimension, and 4.67 microns in its third longest dimension.

A SEM Image of Catalyst B was created. One or more crystal particles with the following three-dimensional size in microns, at least 51 microns for the longest dimension, at least 6 microns for the second longest dimension, and at least 3 microns for the third longest dimension. More specifically, the Catalyst B SEM image shows at least one crystal particle observed to have the following three-dimensional crystal particle size with 51.42 microns as its longest dimension, 6.57 microns in its second longest dimension, and 3.14 microns in its third longest dimension.

A SEM Image was created for Catalyst C. Catalyst C clearly a nano MFI. One or more crystal particles with the following three-dimensional size in microns less than 0.25 microns for the longest dimension, less than 0.25 microns for the second longest dimension, and less than 0.25 microns for the third longest dimension.

A SEM Image was created for Catalyst D. One or more crystal particles with the following three-dimensional size in microns less than 5 microns for the longest dimension, less than 1.8 microns for the second longest dimension, and less than 1.5 microns for the third longest dimension.

More specifically, the Catalyst D SEM Image shows at least one crystal particle observed to have the following three-dimensional crystal particle size with 4.78 microns as its longest dimension, 1.74 microns in its second longest dimension, and very thin, less than 1.5 microns in its third longest dimension.

Without bound to any other theory, one skilled in the art would easily deduce that the present invention is reporting an MFI morphology in three dimensions with a size critical range for high paraxylene selectivity in the product. Catalysts A and B with large crystal sizes in all three dimensions are far superior in achieving high paraxylene concentration in the product compared to the MFIs with smaller dimensions, as exemplified in Catalysts C and D.

### Example 4

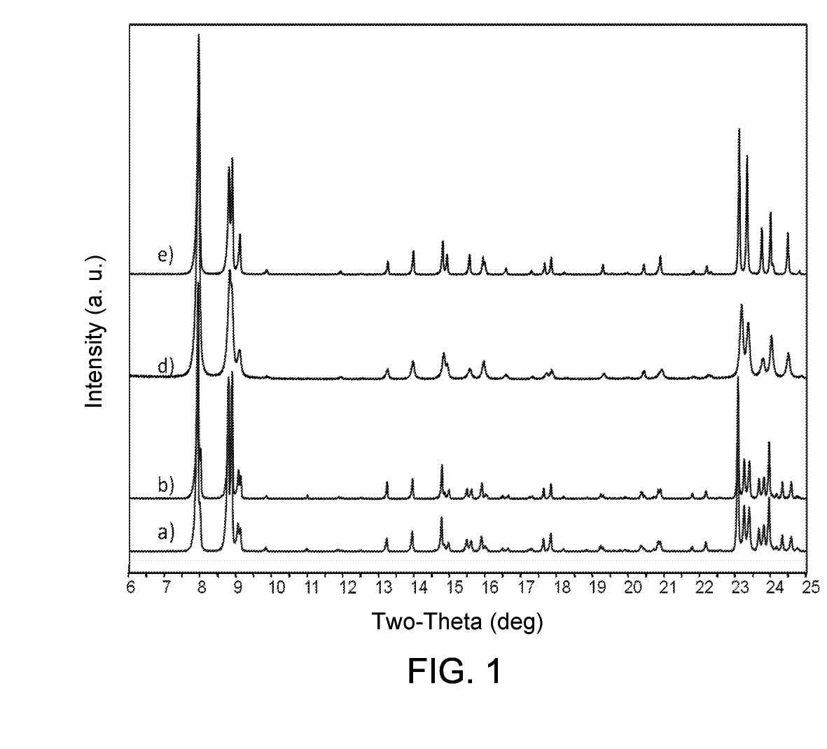

X-ray diffraction data was collected for Catalysts A, B, C, and D in high resolution mode on the Rigaku Smartlab diffractometer in the two-theta range 5-90 deg, equipped with CALSA monochromator with linear PSD. FIG. 1 depicts the X-ray intensity versus two-theta range for Catalysts A, B, C, and D. Catalysts A and B crystal structure is determined to be monoclinic MFI. On the other hand, Catalysts C and D crystal structure is orthorhombic MFI.

TABLE 2
Crystallite Length Results of the Rietveld Refinements

| | Crystallite Length (nm) | | | Strain (%) | | |
|---|---|---|---|---|---|---|
| Material | L₁₀₀ | L₀₁₀ | L₀₀₁ | e0₁₀₀ | e0₀₁₀ | e0₀₀₁ |
| Catalyst A | 494 | 510 | 425 | 3.8 | 2.9 | 2.4 |
| Catalyst B | 412 | 430 | 283 | 5.6 | 3.8 | 3.8 |
| Catalyst C | 123 | 102 | 147 | 7.6 | 6.1 | 6.0 |
| Catalyst D | 374 | 371 | 344 | 1.9 | 1.7 | 2.9 |

Table 2 presents the crystallite length results of the Rietveld Refinements. Without bound to any theory, for the high resolution X-ray diffractometer, Rigaku Smartlab, one skilled in the art in the area of crystallography will be able to infer that deduce that (assuming an increase in peak width of 10% due to crystallite size broadening can be detected) the estimated largest crystallite size that can be measured for Rigaku Smartlab would be about 450 nm. Without bound to any other theory, a crystal size as listed in Table 2 close to and above 450 nm can actually be larger than 450 nm.

Table 2 bulk analysis through X-ray diffraction analysis still shows that Catalyst A has the largest crystal size in all three dimensions. Catalyst B crystal size in the third largest dimension is shorter than Catalyst A. Without bound to any other theory, this observation may explain why Catalyst B shows slightly lower paraxylene selectivity compared to Catalyst A in the performance test results as described in Example 2, Table 1. Clearly Catalysts C and D have much lower crystal dimensions in all three directions. As Table 1 shows, Catalysts C and D show close to equilibrium, that is to say very low, less than 30%, paraxylene concentration in the total xylenes.

In this paragraph, the present invention discloses more details on obtaining the Table 2 results. Looking at Table 2, one can deduce that peak broadening due to strain is negligible in all. The crystallite lengths presented in Table 2 were measured by applying Rietveld refinements to the X-ray diffraction data. The diffraction data collected on a Rigaku Smartlab diffractometer as detailed earlier. The refinement software used was TOPAS (version 4.2, Bruker AXS, 2009). Peak shape was modeled using the fundamental parameters approach with appropriate settings for the Rigaku Smartlab diffractometer. Parameters varied for the Rietveld refinements were the overall scale factor, background (Chebyshev polynomial with 10 coefficients), specimen displacement, anisotropic crystallite size broadening, anisotropic strain broadening, lattice parameters, overall temperature factor, and atomic parameters with weighted constraints. The anisotropic crystallite size broadening and anisotropic strain broadening were modeled using published methods [A. Katerinopoulou, T. Balic-Zunic and L. F. Lundegaard, “Application of the ellipsoid modeling of the average shape of nanosized crystallites in powder diffraction,” J. APPL. CRYST. 45 (2012) 22; P. W. Stephens, “Phenomenological model of anisotropic peak broadening in powder diffraction,” J. APPL. CRYST. 32 (1999) 281].

Catalysts A, B, C, and D were analyzed using IR spectroscopy. The samples were ground to a fine powder. Approximately 10 mg of the powder was weighed out and then pressed into 13 mm diameter self-supporting pellet.

### Example 5

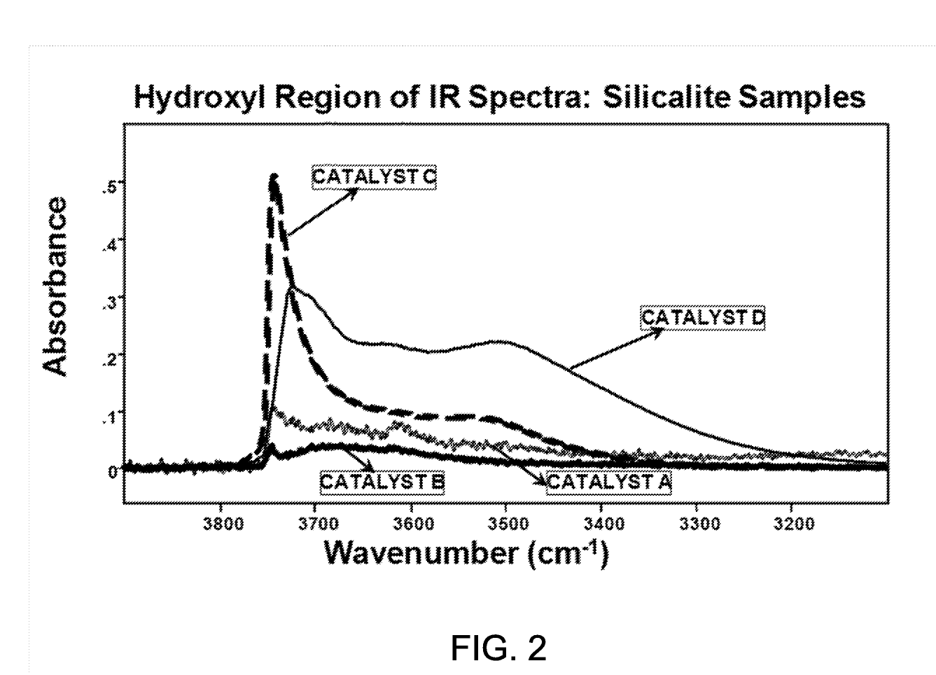

FIG. 2 depicts the results in the OH-IR Region Spectra for Catalysts A, B, C, and D. IR spectra in the OH region measured at room temperature. One skilled in the art will immediately recognize the key differences in the OH range spectra between Catalysts A and B versus Catalysts C and D. Catalysts C and D have no practical application according to the present invention.

TABLE 3
Integrated Area Per Milligram of Sample in the OH-IR Region,
Near 3750 cm-1, Which is Typically Associated With Surface
Silanol “Si—OH” Groups

| | Hydroxyl IR |
|---|---|
| Sample | Si—OH |
| Catalyst A | 0.3840 |
| Catalyst B | 0.0403 |
| Catalyst C | 1.6623 |
| Catalyst D | 1.2079 |

### Example 6

This section discusses IR results obtained with ammonia adsorption for Catalysts A, B, C, and D. Ammonia adsorption was conducted after a pretreatment in helium gas at 500° C. for 2 hours. Ammonia adsorption was performed at 150° C. for 1 hour. Discrete desorption was performed with helium gas flow at 150° C., 300° C. and 400° C. for 1 hour each. All IR spectra were measured at room temperature after each step. The proportions of weak, medium and strong acidity were obtained by applying the following subtractions of integrated peak areas of spectra obtained after discrete desorption steps:

Area(after desorption 150 C)−Area(after desorption at 300 C)=Weak acid sites

Area(after desorption 300 C)−Area(after desorption at 400 C)=Moderate acid sites

Area(after desorption at 400 C)=Strong acid sites

TABLE 4
Integrated Area per Milligram of Sample in the NH₃ IR Characterization

| | Ammonia Adsorption IR | | | | | | | | |
|---|---|---|---|---|---|---|---|---|---|
| | Bronsted (Area/mg) | | | | Lewis (Area/mg) | | | | |
| Sample | Weak | Moderate | Strong | Total | Weak | Moderate | Strong | Total | Sum Total |
| Catalyst A | 0.0656 | 0.0006 | 0.0006 | 0.0668 | 0.0027 | 0.0025 | 0 | 0.0052 | 0.072 |
| Catalyst B | 0.0132 | 0.0056 | 0 | 0.0188 | 0 | 0 | 0 | 0 | 0.0188 |
| Catalyst C | 0.0181 | 0.0022 | 0.0007 | 0.021 | 0.0068 | 0.0039 | 0.0036 | 0.0143 | 0.0353 |
| Catalyst D | 0.0235 | 0.0015 | 0 | 0.025 | 0.0234 | 0.0158 | 0 | 0.0392 | 0.0642 |

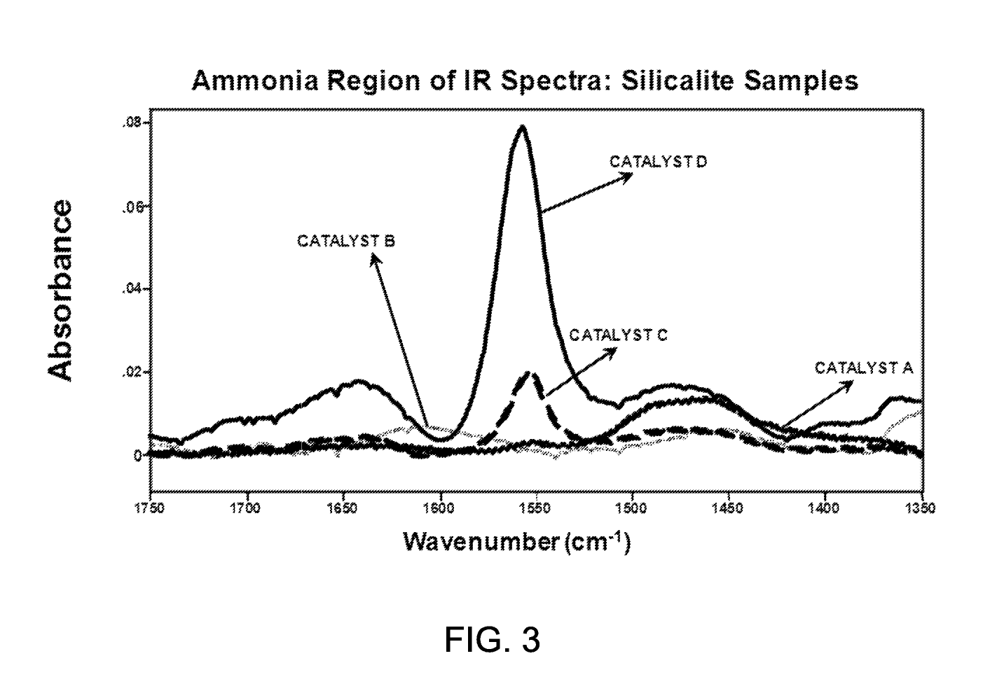

Table 4 provides the NH₃-IR data and the distribution between acidity type and strength. FIG. 3 depicts an example of NH₃-IR spectra taken after one of the three desorption temperatures used to classify the acid strength as weak, medium, and strong. FIG. 3 shows in the region between 1600 to 1500 wavenumber cm-1, Catalysts C and D show distinct strong peaks which are not present in Catalysts A and B. This indicates the existence of more acidic sites in Catalysts C and D that interact with NH₃ in a different way. This evidence of type of interaction is not existent in Catalysts A and B.

One skilled in the art looking at Table 4 will immediately recognize the following. The quantity of different acid sites of Catalysts A, B, C, and D are expressed in Table 4 in units of peak area per mg of material. Table 4 allows one to calculate the distribution of all the acid sites general (Bronsted and Lewis) and allows one to also calculate the acid strength distribution for each acidity type (Bronsted and/or Lewis) as strong, moderate and strong sites. Distribution of the total acidity as Bronsted and Lewis sites: See table 4

Sample A according to the invention has its Bronsted acidity constituting at least 90% of its total acidity. Among the Bronsted acid sites present in Sample A, more than 98% are weak sites and moderate and strong sites represent less than 1% each.

For Sample B, the Bronsted acid sites constitute 100% of the total number of acidic sites where among the 100% Bronsted acidity, 70% are weak Bronsted acid sites and 30% are moderate Bronsted acid sites.

On the other hand, for Sample C (nano MFI2000), the Bronsted acid sites constitute about 59.49% of the total number of acidic sites. Almost 86% of those Bronsted acid sites are weak with about 10% moderate and 3% strong Bronsted acid sites

For Sample D (commercial Tosoh), the Bronsted acid sites constitute about 40% of the total number of acidic sites with around 94% of those as weak Bronsted acid sites and around 6% as moderate Bronsted acid sites.

The Lewis acid sites are distributed as shown in Table 4. Sample A, according to the invention, has its Lewis acidity constituting at most 10% of its total acidity (about 7% in reality) and among the Lewis acid sites present, there is no presence of strong Lewis sites.

Sample B does not contain any Lewis acid sites.

Sample C has its Lewis acidity constituting about 40% of its total acidity, and among the Lewis acid sites present strong and moderate sites represent about 25 and 27% respectively.

Sample D does not contain strong Lewis sites knowing that the total number of Lewis sites in this sample (weak+moderate+strong) constitutes about 60% of the total number of acid sites (Lewis+Bronsted). The Lewis sites distribution is only weak and moderate with close proportions (60 to 40). Compared to the Lewis acid sites distribution in Sample A, this commercial sample has close distribution of its Lewis acid sites with the difference that they are much more abundant compared to Sample A.

Sample B has no Lewis acidity (100% Bronsted) but the distribution of Bronsted sites is 70% weak to 30% moderate. Compared to Sample A, the distribution is larger and also the total number of acid sites is much less (0.02 compared to 0.07). This difference between the two samples according to the invention is resulting from the F/Si ratio (1.6 for Sample A and 0.1 for Sample B).

Sample D, not according to the present invention, has interestingly a close Bronsted distribution, meaning more than 90% as weak Bronsted sites, less than 10 as medium sites, and no strong Bronsted sites. But however, the performance of the sample is inferior compared to Sample A because the total Bronsted acidic sites represent only 40% of the total acidity of the sample, whereby Sample A has 98% of its acidity as Bronsted sites.

### Example 7

Catalysts A, E, and F were tested for MeOH and toluene alkylation. Feed 2:1 (mol:mol) Tol:MeOH. H2:HC (HC=Toluene+Methanol)=2 or 4 (mol:mol); WHSV (on 2 gram of active catalyst)=2, reaction pressures 3 or 10 Barg.

Testing details are as follows: 2 grams of catalyst was mixed with 6 grams of SiC. The combined material was loaded in a 19 mm (0.75 inch) ID stainless steel tubular reactor that has a thermowell to measure the catalyst bed temperature. Before catalytic run, the catalyst was activated in a hydrogen stream at 500° C. for 1 hour, and then a mixture of the feed (toluene and methanol) and hydrogen was passed through the catalyst bed. The reaction was carried out at 400° C., 450° C. and 500° C. The reactor pressure was controlled with a reactor effluent BPR at 3 and/or 10 Barg (about 145 psig) maximum. After BPR, the total reactor effluent vapor composition was analyzed through an on-line GC using dual columns having an FID (Innovax column) as well as TCD (GS Q column). The total reactor effluent composition, including water was determined. Any MeOH in the product was accurately detected and measured.

TABLE 5
Example 7 Catalyst A Performance at a Reactor Pressure of 3 Barg (43.5 psig) Up to 23 Hours On Stream

| | | | | | | | Benzene | | |
|---|---|---|---|---|---|---|---|---|---|
| | | | | | | | to total | MeOH | Xylene |
| Time on | Reactor | Reactor | | H2:HC | | p-Xylene | xylene | conversion | in total |
| stream | temp ABT | pressure | | mol | Toluene | in total | mol:mol | eOH | product |
| hrs | ° C. | Barg | WHSV | mol:mol | conversion % | xylene % | mol ratio | conversion | wt % |
| 1 | 400 | 3.01 | 2 | 2.0 | 17.1 | 82.4 | 0.039 | 99.8 | 7.6 |
| 2 | 400 | 3.01 | 2 | 2.0 | 16.8 | 80.7 | 0.032 | 99.7 | 7.7 |
| 3 | 400 | 3.02 | 2 | 2.0 | 16.8 | 80.7 | 0.026 | 99.7 | 7.6 |
| 4 | 400 | 3.03 | 2 | 2.0 | 15.9 | 79.3 | 0.021 | 99.4 | 7.6 |
| 5 | 400 | 3.05 | 2 | 2.0 | 15.0 | 80.3 | 0.018 | 98.9 | 7.3 |
| 6 | 400 | 3.06 | 2 | 2.0 | 14.2 | 80.3 | 0.016 | 97.7 | 7.2 |
| 7 | 450 | 3.06 | 2 | 2.0 | 15.4 | 77.3 | 0.019 | 93.5 | 10.4 |
| 8 | 450 | 3.07 | 2 | 2.0 | 13.4 | 78.2 | 0.019 | 88.8 | 9.1 |
| 9 | 450 | 3.07 | 2 | 2.0 | 12.5 | 73.2 | 0.018 | 84.8 | 8.4 |
| 10 | 450 | 3.10 | 2 | 2.0 | 10.6 | 74.0 | 0.018 | 80.9 | 7.2 |
| 11 | 450 | 3.10 | 2 | 2.0 | 8.8 | 71.1 | 0.019 | 76.8 | 5.9 |
| 12 | 450 | 3.11 | 2 | 2.0 | 6.5 | 63.8 | 0.023 | 71.9 | 4.6 |
| 13 | 500 | 3.11 | 2 | 2.0 | 12.3 | 67.8 | 0.053 | 85.2 | 7.9 |
| 14 | 500 | 3.11 | 2 | 2.0 | 10.4 | 65.8 | 0.058 | 82.7 | 7.0 |
| 15 | 500 | 3.13 | 2 | 2.0 | 9.5 | 64.4 | 0.062 | 80.8 | 6.4 |
| 16 | 500 | 3.13 | 2 | 2.0 | 8.7 | 63.6 | 0.069 | 79.1 | 5.8 |
| 17 | 500 | 3.15 | 2 | 2.0 | 8.0 | 59.8 | 0.068 | 78.3 | 5.6 |
| 18 | 500 | 3.15 | 2 | 2.0 | 7.3 | 61.0 | 0.074 | 78.8 | 5.0 |
| 19 | 500 | 3.18 | 2 | 4.1 | 6.9 | 65.5 | 0.081 | 70.9 | 4.6 |
| 20 | 500 | 3.19 | 2 | 4.1 | 7.3 | 65.8 | 0.079 | 72.6 | 4.9 |
| 21 | 500 | 3.19 | 2 | 4.1 | 6.9 | 65.9 | 0.077 | 72.8 | 4.7 |
| 22 | 500 | 3.19 | 2 | 4.1 | 7.0 | 65.9 | 0.076 | 73.3 | 4.7 |
| 23 | 500 | 3.19 | 2 | 4.1 | 6.8 | 66.0 | 0.077 | 72.9 | 4.7 |
| 24 | 500 | 1.18 | 2 | 4.1 | 4.3 | 64.5 | 0.088 | 60.5 | 2.9 |
| 25 | 500 | 1.20 | 2 | 4.1 | 3.9 | 62.9 | 0.085 | 63.2 | 2.8 |
| 26 | 500 | 1.22 | 2 | 4.1 | 3.8 | 63.4 | 0.086 | 62.5 | 2.9 |
| 27 | 500 | 1.22 | 2 | 4.1 | 5.0 | 63.5 | 0.086 | 61.5 | 3.1 |
| 28 | 500 | 1.26 | 2 | 4.1 | 3.9 | 65.7 | 0.085 | 64.9 | 2.8 |
| 29 | 500 | 10.22 | 2 | 4.1 | 11.9 | 72.3 | 0.071 | 94.2 | 8.7 |
| 30 | 500 | 10.22 | 2 | 4.1 | 12.0 | 71.7 | 0.070 | 93.8 | 8.9 |
| 31 | 500 | 10.23 | 2 | 4.1 | 12.3 | 71.0 | 0.069 | 92.9 | 8.8 |
| 32 | 500 | 10.22 | 2 | 4.1 | 12.2 | 70.7 | 0.070 | 92.9 | 8.8 |
| 33 | 500 | 10.20 | 2 | 4.1 | 11.9 | 70.0 | 0.071 | 92.0 | 8.7 |

Table 5 provides the key performance indicators when Catalyst A was tested with 2:1 mol:mol toluene to methanol hydrocarbon feed and at operating conditions as provided in Table 5. During the first 23 hours on stream, Catalyst A when tested at a reactor pressure of 3 Barg (43.5 psig) showed a decline in toluene conversion, indicating loss of activity through deactivation.

In MeOH alkylation of toluene for selective paraxylene production, it is desired to keep the benzene in the product low, less than a range from 0.1 to 0.2 benzene to xylene mol:mol. Otherwise, the efficiency of toluene alkylation with MeOH is questionable in the presence of the undesired TDP reaction up to 23 hours on stream. Catalyst A performance in Table 5 indicates that the benzene to xylene mol:mol ratio in the product was less than 0.10, but Catalyst A showed some signs of loss of activity, including a drop in methanol conversion, a drop in pX/X selectivity, and a drop in total xylene weight percent (yield) in the product.

TABLE 6
Example 7 Catalyst A Performance at a Reactor Pressure of 10 Barg (145 psig) Up to 27 Hours On Stream

| | | | | | | | Benzene | | |
|---|---|---|---|---|---|---|---|---|---|
| | | | | | | p-Xylene | to total | | Xylene |
| Time on | Reactor | Reactor | | H2:HC | | in total | xylene | | in total |
| stream | temp ABT | pressure | | mol | Toluene | xylene | mol:mol | MeOH | product |
| hrs | ° C. | Barg | WHSV | mol:mol | conversion % | % | mol ratio | conversion % | wt % |
| 1 | 450 | 10.00 | 2 | 4.1 | 17.4 | 74.1 | 0.081 | 99.7 | 11.1 |
| 2 | 450 | 10.00 | 2 | 4.1 | 17.8 | 73.3 | 0.080 | 99.4 | 11.4 |
| 3 | 450 | 10.00 | 2 | 4.1 | 18.2 | 73.5 | 0.079 | 99.4 | 11.5 |
| 4 | 450 | 10.00 | 2 | 4.1 | 18.0 | 73.1 | 0.078 | 99.4 | 11.4 |
| 5 | 450 | 10.00 | 2 | 4.1 | 18.3 | 73.3 | 0.077 | 99.3 | 11.4 |
| 6 | 450 | 10.00 | 2 | 4.1 | 18.7 | 73.2 | 0.077 | 99.2 | 11.3 |
| 7 | 450 | 10.00 | 2 | 4.1 | 18.3 | 73.7 | 0.077 | 99.4 | 11.2 |
| 8 | 450 | 10.00 | 2 | 4.1 | 18.7 | 73.6 | 0.075 | 99.4 | 11.2 |
| 9 | 450 | 10.00 | 2 | 4.1 | 18.1 | 73.6 | 0.074 | 99.7 | 11.1 |
| 10 | 450 | 10.00 | 2 | 4.1 | 18.2 | 73.9 | 0.075 | 99.1 | 11.1 |
| 11 | 450 | 10.00 | 2 | 4.1 | 18.1 | 73.4 | 0.074 | 99.4 | 11.1 |
| 12 | 450 | 10.00 | 2 | 4.1 | 17.8 | 73.5 | 0.072 | 99.4 | 11.1 |
| 13 | 450 | 10.00 | 2 | 4.1 | 17.9 | 73.6 | 0.072 | 99.3 | 11.0 |
| 14 | 500 | 10.00 | 2 | 4.1 | 18.3 | 68.1 | 0.149 | 99.4 | 13.4 |
| 15 | 500 | 10.00 | 2 | 4.1 | 18.5 | 67.7 | 0.144 | 99.5 | 13.7 |
| 16 | 500 | 10.00 | 2 | 4.1 | 18.8 | 67.7 | 0.142 | 99.4 | 14.1 |
| 17 | 500 | 10.00 | 2 | 4.1 | 18.9 | 68.0 | 0.140 | 99.4 | 14.1 |
| 18 | 500 | 10.00 | 2 | 4.1 | 19.1 | 67.8 | 0.138 | 99.4 | 14.3 |
| 19 | 500 | 10.00 | 2 | 4.1 | 19.4 | 67.6 | 0.133 | 99.5 | 14.6 |
| 20 | 500 | 10.00 | 2 | 4.1 | 19.3 | 67.2 | 0.156 | 99.6 | 14.5 |
| 21 | 500 | 10.00 | 2 | 4.1 | 19.6 | 66.6 | 0.177 | 99.5 | 14.7 |
| 22 | 500 | 10.00 | 2 | 4.1 | 20.0 | 66.6 | 0.160 | 99.6 | 14.9 |
| POWER LOSS | | | | | | | | | |
| POWER LOSS | | | | | | | | | |
| POWER LOSS | | | | | | | | | |
| 26 | 500 | 10.00 | 2 | 4.1 | 18.7 | 67.2 | 0.148 | 99.8 | 14.0 |
| 27 | 500 | 10.00 | 2 | 4.1 | 19.0 | 66.9 | 0.145 | 99.6 | 14.4 |

Table 6 shows the Catalyst A performance when tested at a reactor pressure of 10 Barg (145 psig) up to 22 hours on stream and recovered successfully an unplanned power loss in the experimental facility of the present invention. Performance points marked as 26, 27 hours on stream were obtained after the power was restored, the recovery was good. Most importantly, Catalyst A methanol conversion, xylene yield, toluene conversion was stable. Paraxylene to xylene selectivity remained high and stable. Benzene to xylene mol:mol ratio was less than 0.2, which is acceptable.

TABLE 7
Example 7 Catalyst A Compared to Catalyst E and F

| | | | | | | | Benzene | | |
|---|---|---|---|---|---|---|---|---|---|
| | | | | | | | to total | | Xylene |
| | Reactor | Reactor | | H2:HC | | p-Xylene | xylene | | in total |
| | temp ABT | pressure | | mol | Toluene | in total | mol:mol | MeOH | product |
| Catalyst | ° C. | Barg | WHSV | mol:mol | conversion % | xylene % | mol ratio | conversion % | wt % |
| A | 450 | 10.00 | 2 | 4.1 | 17.9 | 73.5 | 0.073 | 99.4 | 11.1 |
| A | 500 | 10.00 | 2 | 4.1 | 19.6 | 66.8 | 0.164 | 99.5 | 14.7 |
| F | 450 | 3.11 | 2 | 4.1 | 18.8 | 82.6 | 0.063 | 97.9 | 14.2 |
| F | 501 | 3.11 | 2 | 4.1 | 15.9 | 79.6 | 0.163 | 99.5 | 12.6 |
| F | 501 | 10.30 | 2 | 4.1 | 17.2 | 67.1 | 0.395 | 100.0 | 11.1 |
| F | 400 | 2.97 | 2 | 2.0 | 23.1 | 70.8 | 0.098 | 99.7 | 9.6 |
| F | 450 | 2.98 | 2 | 2.0 | 26.2 | 70.6 | 0.064 | 98.9 | 14.2 |
| F | 500 | 2.98 | 2 | 2.0 | 26.8 | 68.0 | 0.123 | 98.7 | 14.8 |
| E | 350 | 3.00 | 2 | 2.0 | 40.6 | 24.7 | 0.036 | 99.9 | 9.8 |
| E | 401 | 3.00 | 2 | 2.0 | 43.9 | 24.0 | 0.068 | 100.0 | 12.7 |
| E | 446 | 3.00 | 2 | 2.0 | 47.0 | 23.5 | 0.073 | 100.0 | 18.7 |
| E | 501 | 3.00 | 2 | 2.0 | 46.3 | 23.3 | 0.137 | 100.0 | 23.1 |

Catalyst A (MF-2000 LC);
Catalyst E (MFI-280);
Catalyst F (MFI-280 X6)

Table 7 shows the performance difference between Catalyst A compared to Catalysts E and F. Catalyst E is the acidic MFI with a 280 oxide ratio. Catalyst E is the parent material for Catalyst F. Table 7 shows that as expected, Catalyst E has close to equilibrium, 24%, which is undesirably low paraxylene in weight percent of total xylene in the product. Catalyst F, in which is the amorphous silica selectivity form of Catalyst E, on the other hand has very high benzene to xylene mol:mol ratio, close to 0.4, which is not a performance that can be considered as acceptable or preferred over for example Catalyst A. Catalyst A can take advantage of higher stability by being able to run at high pressures, in a range from 3 to 50 Barg (45 psig to about 725 psig) with acceptable low levels of benzene in the product, while also maintaining high conversion in both toluene and methanol conversion and stable also high yield in total xylene, and high paraxylene content in the product xylenes.

The examples provided have been intended to demonstrate the main aspects of the present invention and should not be interpreted as limiting examples.

It should be noted that various changes and modifications to the presently preferred embodiments described herein will be apparent to those skilled in the art. Such changes and modifications may be made without departing from the spirit and scope of the present subject matter and without diminishing its attendant advantages.

### SPECIFIC EMBODIMENTS

While the following is described in conjunction with specific embodiments, it will be understood that this description is intended to illustrate and not limit the scope of the preceding description and the appended claims.

A first embodiment of the invention is a process for producing paraxylene comprising alkylating a toluene stream and a methanol stream in a toluene methylation zone operating under toluene methylation conditions in the presence of a catalyst comprising an MFI crystal, alone or bound to any another material, to produce a toluene methylation product stream. An embodiment of the invention is one, any or all of prior embodiments in this paragraph up through the first embodiment in this paragraph, wherein the catalyst includes MFI crystals with a framework silica to alumina ratio of about 50 to about 10,000, more preferably about 100 to about 6,000, or even more preferably about 500 to about 3,000. An embodiment of the invention is one, any or all of prior embodiments in this paragraph up through the first embodiment in this paragraph, wherein the toluene methylation conditions include a temperature of about 250° C. to about 750° C., more preferably between about 350° C. and about 650° C., even more preferably between about 400° C. and about 600° C. An embodiment of the invention is one, any or all of prior embodiments in this paragraph up through the first embodiment in this paragraph, wherein the toluene methylation conditions include a pressure of about 1 Barg to about 100 Barg, more preferably between about 1 Barg to about 50 Barg, even more preferably between 2 Barg to about 30 Barg. An embodiment of the invention is one, any or all of prior embodiments in this paragraph up through the first embodiment in this paragraph, wherein the toluene methylation product stream has a benzene to total xylene molar ratio of less than 1, or preferably less than 0.5, or more preferably less than 0.1. An embodiment of the invention is one, any or all of prior embodiments in this paragraph up through the first embodiment in this paragraph, wherein the catalyst includes MFI crystals with a framework silica to alumina ratio of 2000. An embodiment of the invention is one, any or all of prior embodiments in this paragraph up through the first embodiment in this paragraph, wherein the toluene methylation conditions include a pressure of 3 Barg. An embodiment of the invention is one, any or all of prior embodiments in this paragraph up through the first embodiment in this paragraph, wherein the catalyst is comprised of MFI crystals whose sizes include a first largest dimension, a second largest dimension, and a third largest dimension wherein the first largest dimension is at least 20 microns. An embodiment of the invention is one, any or all of prior embodiments in this paragraph up through the first embodiment in this paragraph, wherein the MFI crystal size includes a second dimension of at least 30% of the first dimension. An embodiment of the invention is one, any or all of prior embodiments in this paragraph up through the first embodiment in this paragraph, wherein the MFI crystal size includes a third dimension of at least 30% of the second dimension. An embodiment of the invention is one, any or all of prior embodiments in this paragraph up through the first embodiment in this paragraph, wherein the process achieves a paraxylene to xylene selectivity of at least 80%. An embodiment of the invention is one, any or all of prior embodiments in this paragraph up through the first embodiment in this paragraph, wherein the process achieves about 15% toluene conversion. An embodiment of the invention is one, any or all of prior embodiments in this paragraph up through the first embodiment in this paragraph, wherein the process achieves a net total xylene yield of close to 15%.

A second embodiment of the invention is a catalyst comprising MFI crystals whose sizes include a first largest dimension, a second largest dimension, and a third largest dimension wherein the first largest dimension is at least 20 microns. An embodiment of the invention is one, any or all of prior embodiments in this paragraph up through the second embodiment in this paragraph, wherein the catalyst includes MFI crystals with a framework silica to alumina ratio of about 50 to about 10,000, more preferably about 100 to about 6,000, or even more preferably about 500 to about 3,000. An embodiment of the invention is one, any or all of prior embodiments in this paragraph up through the second embodiment in this paragraph, wherein the composition includes at least 60% of the acid sites are Bronsted acid sites. An embodiment of the invention is one, any or all of prior embodiments in this paragraph up through the second embodiment in this paragraph, wherein at least 70% of the Bronsted acidity is present as weak acid sites. An embodiment of the invention is one, any or all of prior embodiments in this paragraph up through the second embodiment in this paragraph, wherein among the Bronsted acid sites, less than 5% of those exist as strong acid sites. An embodiment of the invention is one, any or all of prior embodiments in this paragraph up through the second embodiment in this paragraph, wherein the MFI crystal size includes a second dimension of at least 30% of the first dimension. An embodiment of the invention is one, any or all of prior embodiments in this paragraph up through the second embodiment in this paragraph, wherein the MFI crystal size includes a third dimension of at least 30% of the second dimension.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for producing paraxylene comprising alkylating a toluene stream and a methanol stream in a toluene methylation zone operating under toluene methylation conditions in the presence of a catalyst comprising an MFI crystal, alone or bound to any another material, to produce a toluene methylation product stream.

2. The process according to claim 1, wherein the catalyst includes MFI crystals with a framework silica to alumina ratio of about 50 to about 10,000, more preferably about 100 to about 6,000, or even more preferably about 500 to about 3,000.

3. The process according to claim 1, wherein the toluene methylation conditions include a temperature of about 250° C. to about 750° C., more preferably between about 350° C. and about 650° C., even more preferably between about 400° C. and about 600° C.

4. The process according to claim 1, wherein the toluene methylation conditions include a pressure of about 1 Barg to about 100 Barg, more preferably between about 1 Barg to about 50 Barg, even more preferably between 2 Barg to about 30 Barg.

5. The process according to claim 1, wherein the toluene methylation product stream has a benzene to total xylene molar ratio of less than 1, or preferably less than 0.5, or more preferably less than 0.1

6. The process according to claim 2, wherein the catalyst includes MFI crystals with a framework silica to alumina ratio of 2000.

7. The process according to claim 4, wherein the toluene methylation conditions include a pressure of 3 Barg.

8. The process according to claim 1, wherein the catalyst is comprised of MFI crystals whose sizes include a first largest dimension, a second largest dimension, and a third largest dimension wherein the first largest dimension is at least 20 microns.

9. The process according to claim 8, wherein the MFI crystal size includes a second dimension of at least 30% of the first dimension.

10. The process according to claim 8, wherein the MFI crystal size includes a third dimension of at least 30% of the second dimension.

11. The process according to claim 1, wherein the process achieves a paraxylene to xylene selectivity of at least 80%.

12. The process according to claim 1, wherein the process achieves at least 15% toluene conversion.

13. The process according to claim 1, wherein the process achieves a net total xylene yield of at least 10%.

14. A catalyst comprising MFI crystals whose sizes include a first largest dimension, a second largest dimension, and a third largest dimension wherein the first largest dimension is at least 20 microns.

15. The catalyst according to claim 14, wherein the catalyst includes MFI crystals with a framework silica to alumina ratio of about 50 to about 10,000, more preferably about 100 to about 6,000, or even more preferably about 500 to about 3,000.

16. The catalyst according to claim 14, wherein the composition includes at least 60% of the acid sites are Bronsted acid sites.

17. The catalyst according to claim 14, wherein at least 70% of the Bronsted acidity is present as weak acid sites.

18. The catalyst according to claim 14, wherein among the Bronsted acid sites, less than 5% of those exist as strong acid sites.

19. The catalyst according to claim 14, wherein the MFI crystal size includes a second dimension of at least 30% of the first dimension.

20. The catalyst according to claim 14, wherein the MFI crystal size includes a third dimension of at least 30% of the second dimension.
